(19) United States
(12) Patent Application Publication
(10) Pub. No.: US 2008/0227993 A1
(43) Pub. Date: Sep. 18, 2008

(54) Synthesizing and compounding molecules from and with plant oils to improve low temperature behavior of plant oils as fuels, oils and lubricants

(76) Inventor: Matthew Mark Zuckerman, Toluca Lake, CA (US)

Correspondence Address: GEORGE S. COLE, ESQ., 495 SEAPORT COURT, SUITE 101, REDWOOD CITY, CA, 94063, US

(21) Appl. No.: 11/725,254

(22) Filed: Mar. 17, 2007

(51) Int. Cl. C07C 51/285; C07C 59/125

(52) U.S. Cl. 554/149; 554/1; 554/219

(57) ABSTRACT

The present invention is a method for making a class of molecules synthesized from unsaturated plant oils, and the synthesized class of molecules, such that when compounded with saturated plant oils they improve the physical properties such as low temperature behavior, measured as cold filter plug point and cloud point for biodiesel fuels and pour point for oils and lubricants, as well as other physical properties including viscosity and viscosity index, so that the physical properties of the combined materials approach the physical properties of unsaturated plant oils and find use as base material feed stocks for “Green” fuel, oil, and lubricant products.

### BACKGROUND OF THE INVENTION

Today most fuels, oils and lubricants are produced from a feed stock of crude oil, that is, from the class of hydrocarbons called mineral oils. Similar products produced from feed stocks such as palms and soybeans are from the class called plant oils. Unlike those produced from mineral oils, fuels, oils and lubricants based on plant oils are generally rapidly biodegradable, of low ecotoxicity, and come from a renewable resource. One objective of our nation—recently recognized as being of increasing priority—is reducing our reliance on crude oil; one way to help meet this objective is to source an increasing percentage of the supply of fuels, oils, and lubricants from plant oils. Unfortunately, the demand for fuel is so tremendous that supplying the feed stocks to make a single-digit percentage of the national consumption of this product from plant oils taxes current agricultural capabilities. When the current US consumption of diesel fuel for on-road uses is 40 billion gallons a year, and (it is estimated) when planting every acre possible with soybeans will produce only one billion gallons of diesel, the deficit is obvious—and this also falls far short of the government's objective of producing six billion gallons of ‘biodiesel’ in 2010.

Fuel is not the only product currently produced using mineral oil based hydrocarbons; so, too, are oils and lubricants. (The potential interchangeability probably dates back to merchants in the Classical and Fertile Crescent civilizations swapping between using pig grease and ‘napthum’ on wood-axled carts.) But modern oils and lubricants have far more particular, or at least understood, requirements; requirements that to date have favored mineral-oil based over plant-oil based products.

The most common sources for plant oils are corn, soybean, palm, rapeseed (canola), sunflower. Corn and soybean oils are used for ethanol fuel production, which puts upward pressure on the price of these feed stocks and reduces the quantities available for biodiesel fuel and oils and lubricants. The plant oils that are available in quantities at a price that makes them economically feasible are first palm and second soybean.

The term ‘cloud point’ describes the temperature when a biodiesel is cooled to where a change of state from liquid to solid first starts to occur, because a cloud becomes visible in the liquid. This change of state also clogs the fuel filter for diesel engines, and thus the ‘cloud point’ also indicates the temperature below which the filter clogs (and so is also known as the cold filter plug point). Lowering the cold filter plug point for a given biodiesel lubricant below the lowest ambient temperature encountered at any time in the year at a particular local allows year-round use of that biodiesel and avoids the cost of cleaning the filter so that the engine can receive the fuel. This behavioral change due to an incipient state-change at low temperatures, expressed as the cold filter plug point temperature for diesel fuel, is called the ‘pour point’ for oils and lubricants.

Biodiesel fuel entirely made from a feed stock of soybeans has a cloud point of zero degrees Centigrade—the temperature at which water freezes. This is a temperature that almost every American state (excepting Hawaii), and the great majority of the national territory, experiences during a lesser or greater part of the year. While an engine usually maintains a higher temperature while operating (unless experiencing Alaskan-style winter temperatures and/or extreme evaporative ‘wind chill’ cooling), it is both hazardous and not fuel-efficient to keep any engine continuously running day-and-night throughout even a short ‘cold snap’. Thus the expected minimal ambient temperature is a critical concern for any fuel, oil, or lubricant; and a sub-zero-Centigrade ‘cloud point’ or ‘pour point’ is almost a necessity.

Palm oil, which has a higher percentage of saturated fatty acids than soy oil, has an even higher cloud point—five to seven degrees Centigrade. The plant oil compound that to date has exhibited the best low-temperature behavior contained 90% oleic acid, and had a pour point of −40 degrees Centigrade, was formed with 18-carbons and one double bond, and was obtained from high oleic sunflower oil. Generally good to excellent low temperature behavior has also been found in short-chain fatty acids with five to nine carbon chain lengths; but the intermediate carbon chain lengths exhibit worsening low-temperature behavior.

Palm oil has almost a tenfold greater yield of oil per acre, and sufficient acreage is being planted or is currently planned, to supplement soybean-based biodiesel in order to help reach the goal of reducing our reliance on imported (mineral) oil. Unfortunately palm oil is disadvantageous as a feed stock because it contains a substantial quantity of saturated oils. These account for more than half the weight and are principally palmitic acid and to a lesser amount, stearic acid. Palmitic acid in particular has a poor ‘low temperature’ property, for it is solid at room temperature. At present palm oil is chiefly seen as best used in food preparation (as a substitute for lard, for example), or in soap.

The most important fatty acids contained in plant oils are the saturated and unsaturated fatty acids. Fatty acids consist of the elements carbon (C), hydrogen (H), and oxygen (O) arranged as a carbon chain with a carboxyl group (—COOH) at one end. Saturated fatty acids have all the hydrogen that the carbon atoms can hold, and therefore have no double bonds between the carbons. Monosaturated fatty acids have only one double bond. Polyunsaturated fatty acids have more than one double bond.

The common fatty acids have both common and scientific names. The numbers at the beginning of the scientific names indicate the location(s) of the double bonds, with (by convention) the carbon of the carboxyl group being carbon number one. For example, the 4-carbon, zero-double bond fatty acid with the common name of ‘butyric acid’ has the scientific name of ‘butanoic acid’. Butyric/butanoic acid is one of the saturated short-chain fatty acids, is responsible for the characteristic flavor of butter, has the equivalent line formulas of CH₃CH₂CH₂COOH or CH₃(CH₂)₂COOH, and is a carbon chain where one end carbon has three bonds with hydrogen atoms, the middle two carbons each have two separately bonded hydrogen atoms, and the other end carbon has a double bond with on oxygen atom and a single bond to an OH group (thus this carbon, the two oxygen, and one hydrogen atom form the —COOH carboxyl group). While describing butyric/butanoic acid in text or even in a line formula is manageably readable (though providing eyestrain and finger-counting for the text proofer), this is less true for the longer carbon-chain structures. For example, linoleic acid has the scientific name of 9,12-octadecadienoic acid. (‘Octa’ ‘deca’ or 8 10 in Greek giving the 18 carbon chain; ‘di’ ‘en’ or 9 12 locating the double bonds at carbons 9 and 12, with ‘oic acid’ showing that carbon 1, by convention, is anchoring the carboxyl group. The structural formula for linoleic acid is: CH₃CH₂CH₂CH₂CH₂CH═CHCH₂CH═CHCH₂CH₂CH₂CH₂CH₂CH₂CH₂COOH; it abbreviates to CH₃(CH₂)₄CH═CHCH₂CH═CH(CH₂)₇COOH. For this reason a shorthand notation such as C18:2 is used to indicate that the fatty acid consists of an 18-carbon chain with 2 double bonds in the locations where they are found in the naturally-occurring fatty acid, i.e. linoleic acid (so the double carbon bonds in a C18:2 are presumed to be at 9, 12 carbons, respectively). Shorthand Latin prefixes Cis and Trans describe the orientation of the saturating hydrogen atoms with respect to the double bond, with Cis meaning “on the same side” and Trans meaning “across” or “on the other side”. Generally, naturally occurring fatty acids have the Cis configuration. Another means of showing a carbon chain is to simply have a zig-zag line where each vertex (up or down point) represents a carbon atom, with double bonds being represented by double and parallel horizontal lines. Finally, a generalized symbol for a ‘fatty acid’ in these chemical formulas is the capital letter ‘R’.

The saturated fatty acids are palmitic, with a 16-carbon chain and no double bonds (C16:0) and stearic, with an 18-carbon chain and no double bonds. The unsaturated fatty acids are oleic, with an 18-carbon chain and one double bond (C18:1); linoleic, with the same carbon chain length and two double bonds (C18:2); and linolenic acid, also with the same carbon chain length and three double bonds (C18:3).

The structure of fatty acids, their chain length and degree of saturation, are directly related to their properties as a fuel, oil or lubricant—this includes their operational stabilities as well as their lubrication properties such as viscosity, viscosity index (sensitivity of viscosity to changes in temperature), and low temperature behavior (cold filter plug point, pour point and cloud point). The oxidative stability of plant oils is inversely related to their compositional percentage of polyunsaturated acids; the oxidative stability increases as the amount of polyunsaturated acids decreases. At least one cis-(Z) double bond is essential to good low-temperature behavior, thus making a high content of oleic acid, or derivatives of oleic acid with a single double bond, is a desirable ingredient in plant-oil based fuel, oils and lubricants. Also, increasing the branching and shortening of the carbon chain length improves (lowers) the pour point. These properties are disclosed in “Review Plant-oil-based lubricants and hydraulic fluids”, Manfred P. Schneider, J. Sci. Food Agric.86:1769-1780, esp. p. 1772; © 2006 Society of Chemical Industry, published online Aug. 3, 2006; DOI: 10,1002/jsfa.2559, herein incorporated by reference.

Generally fuels, oils and lubricants are composed of base materials and additives. Each is also usually a mixture of compounds. The effect of one material in the mixture on another material can be agonistic or antagonistic, and the interplay between the molecules is generally little understood in scientific terms. For example, petroleum-based motor oil is a blend of base materials and contains approximately 10% additives. Additives are generally described by their function, and compounds commonly available exist for a great many varying needs, including among others: antioxidant, metal deactivator, extreme pressure, antifoaming, pour point depressant, anti-icing, corrosion inhibition, detergent-dispersant, and combustion improvement.

Plant oils are base materials that can only be used, straight out of the barrel, for low-performance application without suitable additives. Most existing additives for petroleum-based fuels, oils and lubricants have poor biodegradability and undesirable ecotoxicity. Reducing or eliminating additives in the production of fuels, oils and lubricants from plant oil base materials, and thus preserving the “Green” aspect of the products as far as possible, is both desirable and beneficial.

The approximate composition of high oleic sunflower oil (HOSO) is a concentration of the saturated fatty acids (oleic at 90%, stearic at 2%), with a small fraction of the unsaturated fatty acids (linoleic at 3.5% and no linolenic acid). Plant oils high in oleic acid, and derivatives thereof, are the best feed stock for fuel, oil and lubricants. However, HOSO is a high grade relatively high priced oil, and is not available in quantities that can have a significant impact on the nation's objective of reducing dependence on crude oil. The approximate composition of palm oil is a balanced mix of saturated and unsaturated oils (the saturated oils of palmitic at 40% and stearic at 10% and the unsaturated oils oleic at 40% and linoleic at 10%). In order to reduce our nation's dependence on crude oil what is needed is a compound and the means to make it that enable combining in one material the saturated plant oils contained in palm (palmitic and stearic) and soybean oil and other ingredients that allow the resulting mixture to take on the beneficial properties of the preferred unsaturated oils (esp. oleic & linoleic).

U.S. Pat. No. 6,197,731 (Zehler et al., Mar. 06, 2001), as it title states, discloses base stocks for “Smokeless Two-Cycle Engine Lubricants”. Two-stroke engines, as the prior art shows, mix the lubricant with the fuel (though perhaps varying the proportion as the operating temperature changes). This patent discloses compositions including at least two esters wherein “the second ester comprises polyol residues and polycarboxylic acid residues” (Independent claims 1, 15, and 25), are mineral-oil and not plant-oil based, and thus are not renewably sourced. Furthermore, this invention focuses on the ‘smokeless’ nature of the final product rather than on the sourcing and lubricant functionality of the final composition; with the specification accepting that 0.01-15%, preferably 1-6% (though up to 50% for polybutene), of the final composition may be comprised of “various other additives” which are generally non-biological compounds or solvents (including kerosene).

U.S. Pat. No. 6,656,888 (Zehler, Dec. 02, 2003) also discloses two-cycle lubricants using biodegradable ester base stocks. That patent accepts the test method CEC-L-33-T-82 developed by the Coordinating European Council (CEC) and reported by the CEC in “Biodegradability of Two-Stroke Cycle Outboard Engine Oils in Water: Tentative Test Method,” pp. 1-8, to define what comprised “rapidly” (>70%) and “readily” (>80%) biodegradable materials. Using this test, mineral oils are 15%-30% biodegradable, natural vegetable oils are 70% to 95% biodegradable, and esters are up to 95% biodegradable, depending on chemical structure. This patent discloses use of a ‘grease formulation’ that preferably comprises a “polyol ester which has as its reactive components neopentyl polyol and a C.sub.12-C.sub.20 monocarboxylic acid”, focusing specifically on “”C.sub.12-C.sub.20 branched chain saturated monocarboxylic acids”. However, the composition generally will include a thickening agent (claim 1: “admixed with additive thickener”) which the specification describes as generally being non-organic, with “soaps of lithium, barium, aluminum, calcium and mixtures thereof are the most commonly used”, while “Other thickening agents that may be used according to the invention include inorganic materials such as silica and clay”.

U.S. Pat. No. 6,828,287 (Lakes et al., Dec. 07, 2004) also discloses “Biodegradable Two-Cycle Engine Oil Compositions and Ester Base Stocks”. These ester base stocks are “a neopentylpolyol and a C.sub.16-C.sub.20 branched chain, saturated monocarboxylic acid” (Specification, Independent claims 1, 7), “a neopentylpolyol and a C.sub.16-C.sub.20 straight chain saturated monocarboxylic acid (Ind. Claim 12) or a “neopentylpolyol and a C.sub.8-C.sub.10 straight chain, saturated monocarboxylic acid” (Ind. Claim 20).

At best the compositions described in the above-referenced patents include some mix of mineral and plant oils and are neither, as in the present invention, entirely plant-oil based, nor do they incorporate the unexpected results of improved functionality gained through combining the saturated and unsaturated fatty acids from plant oils disclosed below.

### SUMMARY OF THE INVENTION

The present invention is a method for synthesizing a class of molecules from unsaturated plant oils (such as the methyl form of oleic acid and linoleic acid), which compounded with saturated plant oils (such as derivative forms of palmitic and stearic fatty acids) form a composition whose physical properties approach the beneficial physical properties of pure saturated plant oils (such as oleic and linoleic fatty acids and derivatives thereof), particularly possessing low temperature behavior (measured as cold filter plug point and cloud point for biodiesel fuels and pour point for oils and lubricants), as well as other physical properties of pure unsaturated plant oils, including viscosity and viscosity index, without compromising the beneficial properties of the unsaturated plant oils (such as oxidative stability), whereby the composition can be used as base material feed stock for a broad range of “Green” fuel, oil and lubricant products; that class of molecules; and the method for making such compositions.

The class of molecules in the compositions will contain a stearic acid base with no double bonds, have a structure of one base molecule attached to one or two branched molecules, include zero, one or two hydroxy groups so that the molecule will be both polar and soluble in the fatty acid solution or is an anhydrous form of the polar molecule, while the branched molecule(s) will have a carbon chain of between 5 to 9 length, and will also be saturated with no double bonds.

The organic synthesis to produce these compositions of the class of molecules consists of two or more of the following steps: (a) Epoxidation; (b) Hydrolysis; (c) Esterification; and (d) Ozonolysis. As the quality curve for good pour-point temperatures versus carbon chain length both takes a bell-shape where the best behavior is exhibited in stand-alone extremes chains that are either 5-to-9 carbons or 18-or-longer, optimal combinations for fuel, oils and lubricants will combine oils having a long chain of 18 carbons with those having a range between five and nine carbons, or synthesize them into a compound having both a long chain of 18 carbons and one or two branches with a range between five and nine carbons.

It is an object of the present invention to make a plant-based composition using a portion of oleic acid that, when compounded with saturated oils, will allow for the compounded material to be a base material for fuels, oils and lubricants with low temperature behavior similar to that of fuels, oils and lubricants comprised from pure oleic acid.

It is further an object of the present invention to use plant oils and other materials in the synthesizing process that are entirely not based on mineral oils (crude oil), and thus create a method with superior biodegradability and low ecotoxicity at all stages.

It is further an object of the present invention to reduce the nation's dependence on crude oil by extending the low-temperature capabilities of biodiesel fuels, oils and lubricants making them suitable for year-round use in colder climates and allowing increased supply of feed stocks for these products by using the world's large agriculture supplies of saturated oil fractions of plant oils such as palm oil.

It is further an objective of the present invention to produce a blended base material that will reduce the cloud point and cold filter plug point of biodiesel made from palm, soybean and other plant-oil feed stocks to equal or exceed the cloud point and cold filter plug point of crude oil based diesel fuel.

It is further an object of the present invention for the blended base material to be made from feed stocks that are all plant-oil and not crude-oil based for improved biodegradability and reduced pollution load on the air and water and reduced carbon footprint from the totality of the production and synthesizing cycle.

It is further an object of the present invention to enable the synthesis and blending of plant-oil feed stocks to produce fuel at a cost that will not so burden the price of biodiesel as to be noncompetitive with diesel fuel manufactured from crude oil.

It is still further an object of the present invention to make enable the production of a line of fuel, oil and lubrication products based on palm, soybean and other renewable, plant-based feed stocks, that have superior performance to that of crude oil and other synthetic oil based materials, enabling the former line of products to command a premium price in the low-temperature application markets.

### BRIEF DESCRIPTION OF THE DRAWINGS

FIG. 1A illustrates the first (“TYPE A”) of three structural forms of the class of molecules synthesized according to the method of the present invention.

FIG. 1B illustrates the second (“TYPE B”) of three structural forms of the class of molecules synthesized according to the method of the present invention.

FIG. 1C illustrates the third (“TYPE C”) of three structural forms of the class of molecules synthesized according to the method of the present invention.

FIGS. 2A and 2B illustrate the synthesis of branched methyl linoleate with butyric acid according to the method of the present invention.

FIGS. 3A and 3B illustrate the synthesis of a butyric of methyl oleate according to the method of the present invention.

FIGS. 4A and 4B illustrate the synthesis of a nonanoic of methyl oleate according to the method of the present invention.

FIGS. 5A, 5B and 5C illustrate the synthesis of a methyl oleate with butyric anhydride according to the method of the present invention.

FIG. 6 illustrates as a flow chart the method for producing from an original plant oil source (in this example, commonly commercially available Palm Oil), not only two of the class of molecules synthesized according to the method of the present invention, but also a mixture suitable for producing plant-oil based fuel, oil, and lubricants.

FIG. 7 illustrates as a flow chart the method for producing from an original plant oil source (in this example, commonly commercially available Palm Oil), not only the class of molecules synthesized according to the method of the present invention, but also a specific final product that is a plant-oil based lubricant, wherein the proportions (using alternate blending weights of oleic oil, for example) will determine the specific qualities for a series of grades, in similar fashion to the preparation of lubricants ranging from 10W-30 to 20W-40 motor oil produced from a mineral-oil source stock.

### DETAILED DESCRIPTION OF THE INVENTION

The class of materials that when compounded with saturated acids such as palmitic acid, or forms of palmitic acid used in the application contemplated hereby such as methyl palmitate, are illustrated by the three polar molecules and the one anhydrous form. One skilled in the art of organic chemical synthesis is capable of taking the information provided and not only producing these four materials, but also understanding how logical extension through processes well known to the average practitioner in the art, by substituting other materials in the synthesizing and manufacturing processes, can be used to obtain other final products that may differ from these four molecules yet still fall within the teaching of the present invention as to the structure of the resulting materials, that reproduce the favorable results, such as of improving low temperature behavior, as is claimed herein.

The feed stocks in the examples shown begin with a plant oil based, long carbon chain fatty acid, one or two double carbon bonds, such as methyl oleate (18:1) or methyl linoleate (18:2) that serves as the starting point for synthesis of the desired class of molecules. The chain lengths of the branch or branches shown in the three forms of the class of molecules produced that are embodiments of the present invention are either 5 or 9 carbons. But other desired molecular structure embodiments can be obtained by use of still other desired short carbon chain length molecules with a number of carbons between 5 and 9 without departing from the present invention.

The present invention differs from the referenced US Patents above (Zehler et al., Zehler, and Lakes et al.) specifically in that in those patents the branched molecules have quaternary carbon atoms (carbon atom bonded to four other carbon atoms with single bonds), which in the present invention are not present, as the methyl esters of oleic acid and linoleic acid are attached through the esterification of neopentyl or trimethylol propane which has the primary or terminal hydroxy group. In the present invention a branched molecule with more than one methyl ester fragment can be easily achieved by using these primary alcohols. Further, in the present invention branching is limited by having tertiary carbon atoms and the short chain fatty acids attached through the esterification methyl ester which has secondary hydroxy groups. No such limitation on branching exists in the referenced US patent applications. Lastly the utility of the present invention is directed at improving the low temperature behavior of saturated plant oils, a different goal than that of either of the referenced US patents; although the present invention may find use in production of a similar class of product along with other biodiesel-based fuel, oil and lubrication products.

The class of molecules in the present invention is illustrated by the following four molecules, three of which are synthesized from the methyl form of oleic acid and one from the methyl form of linoleic acid:
methyl 9,12-dihydroxyoctadecanoate 10,13-dibutyrate;
methyl 10-hydroxyoctadecanoate 9-butyrate;
methyl 10-hydroxyoctadecanoate 9-nonanoate; and,
methyl octadecanoate 9,10-dibutyrate.

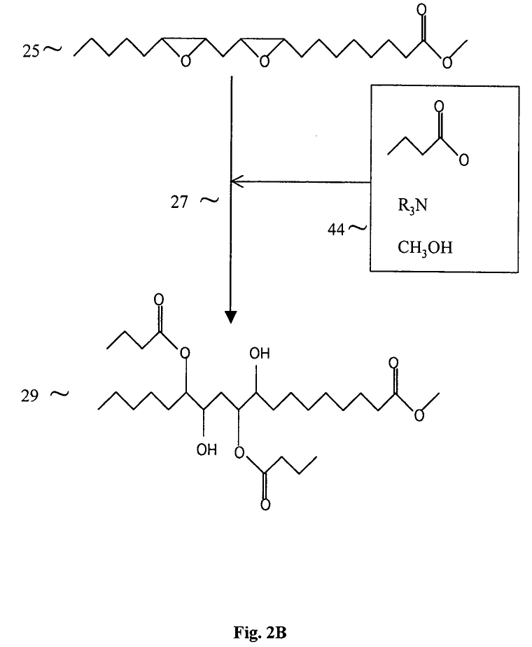

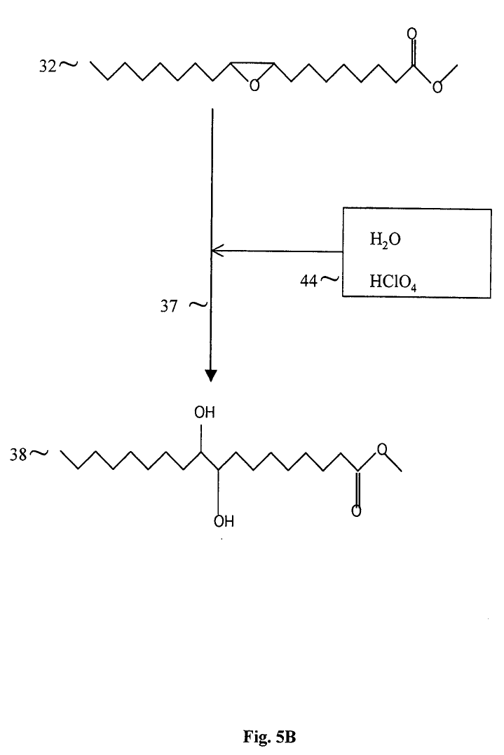

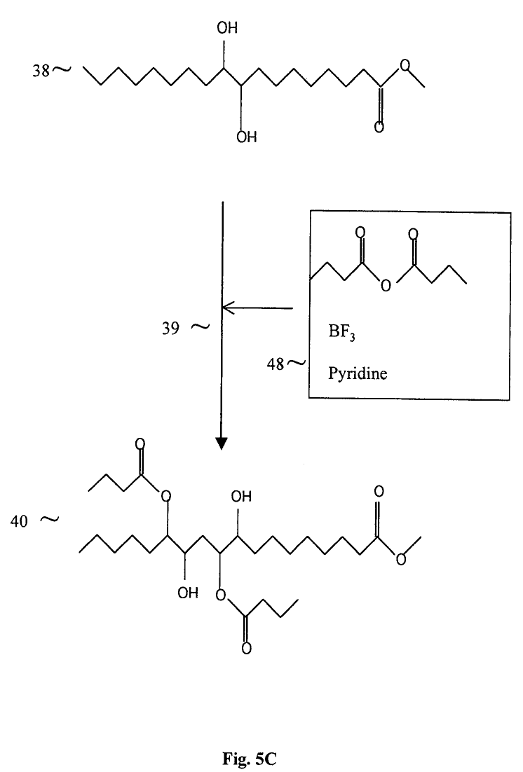

The preferred embodiment of the present invention is methyl 9,12-dihydroxyoctadecanoate 10,13-dibutyrate (FIG. 2B), and the second most preferred embodiment of the present invention is Methyl octadecanoate 9,10-dibutyrate (FIG. 5C). This preference is based on the belief that more branching and polarity are desirable structural properties of the molecule for the present invention, but that it is more important to have a branched molecule or molecules even if that comes at the expense of sacrifice of a hydroxy group or hydroxy groups. For improved low-temperature behavior the presence of hydroxy groups is important; however, it is of secondary import to the high degree of branching.

The organic synthesis for each of the four molecules, which are embodiments of the class of materials in the present invention, requires the use of two or more of the following processes: Epoxidation, Hydrolysis, Esterification and Ozonolysis. The synthesis requires the following: (1) equipment, glassware and supplies; (2) chemicals; and (3) instruments to characterize the synthesized molecules.

1. Equipment, glassware and supplies:
1 L three-neck round bottom flask
Magnetic stirrer hotplate, stir bars and rubber septa
Reflux condenser, Thermometer and Nitrogen inlets
Dropping funnel, Measuring jar
Oil bath or Heating mantle and steam bath
Low temperature source (ice or cold water)
Syringes and needles
Vacuum distillation apparatus or glassware
Vacuum double manifold (to perform the reaction under inert atmosphere)
Vacuum line (vacuum pump is better)
Nitrogen or Argon gas
Accessories (Lab jack, pH indicator strips, glass stopper, vacuum grease, rubber tubing, gloves, clamps and holder)

2. Chemicals:

(a) for Epoxidation:
Methyl oleate or methyl linoleate
Hydrogen peroxide and Formic acid
Diethyl ether, distilled water and Magnesium sulfate

(b) for Hydrolysis
5% KOH and cold HCl (1N) or Perchloric acid
Distilled water and diethyl ether

(c) for Esterification:
Carboxylic acid (butyric, nonanoic or azelaic acid) or anhydride
Tertiary amine (e.g. Et₃N) and Methanol
BF₃ and Pyridine to esterify anhydrides

(d) for Ozonolysis
Oleic acid (to get azelaic and nonanoic acid)
O₃ (ozone) and Methanol
Zn/H₂O

3. Instruments to characterize the synthesized molecule:
Infrared Spectroscopy
NMR Spectroscopy
GC-MS Spectroscopy

### DETAILED DESCRIPTION OF THE DRAWINGS

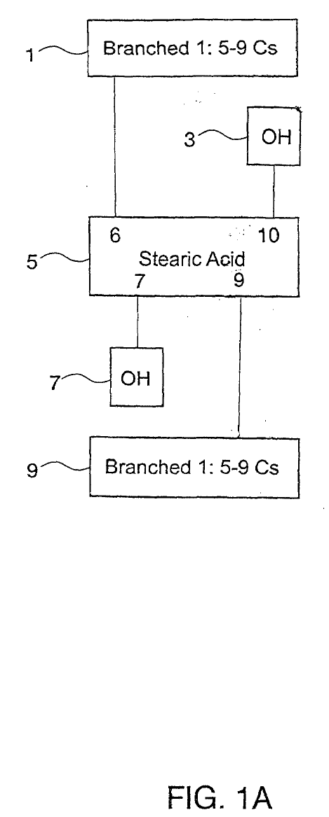

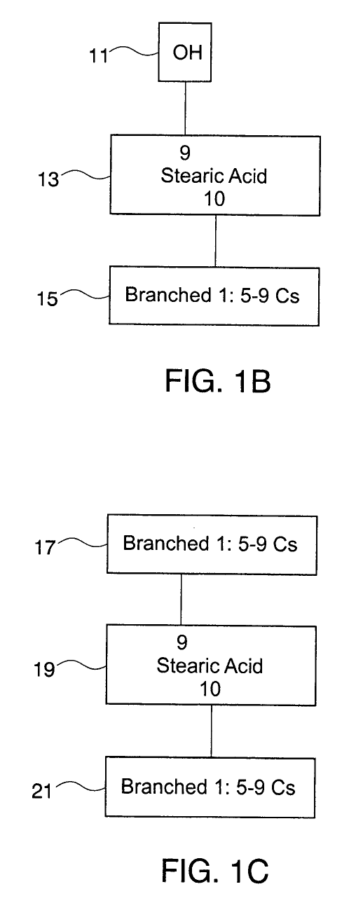

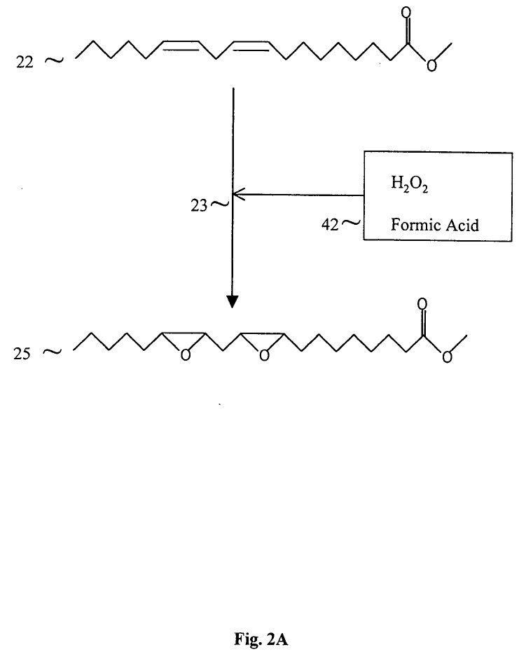

FIGS. 1A, 1B, and 1C respectively illustrate the first, second, and third of three structural forms of the class of molecules that are to be synthesized and form the compositions according to the method of the present invention. FIG. 1A illustrates Type A; FIG. 1B illustrates Type B; and FIG. 1C illustrates Type C, as more specifically described below.

FIG. 1A illustrates the first of three structural forms of the class of molecules that are to be synthesized, Type A. Type A has as a central skeleton [5] a form of stearic acid (scientific name, octadecanoic acid), an 18:0 carbon chain. This molecule differs from pure stearic acid as it also incorporates as part of the core carbon chain not just attached single hydrogen molecules, but a first branch that is a five-to-nine carbon chain fatty acid [1] at carbon 6; a first hydroxy group [7] at carbon 7, a second branch that is also a five-to-nine carbon chain fatty acid [9] at carbon 9, and a second hydroxy group at carbon 10 [3], and thus Type A is a branched methyl linoleate with (preferentially) butyric acid. The preferential form having at both the first branch and second branch a five-carbon chain length fatty acid has a scientific name of methyl 9,12-dihydroxyoctadecanoate 10,13-dibutyrate. This molecule's chemical formula is C₂₇H₅₀O₈, and its structure is:

CH₃(CH₂)₄CH(OCOCH₂CH₂CH₃)CH(OH)CH₂CH(OCOCH₂CH₂CH₃)CH(OH)(CH₂)₇COOCH₃.

FIG. 1B illustrates the second of three structural forms of the class of molecules that are to be synthesized, Type B. Type B has as its central skeleton [13] a form of stearic acid (scientific name, octadecanoic acid), an 18:0 carbon chain. This molecule differs from pure stearic acid as it also incorporates as part of the core carbon chain not just attached single hydrogen molecules, but a hydroxy group [11] at carbon 9 and also a single branch containing a five-to-nine carbon chain length fatty acid [15] at carbon 10. When the single branch is, as a first preference, a five-carbon chain length fatty acid [15] at carbon 10, the resulting molecule's scientific name is methyl 10-hydroxyoctadecanoate 9-butyrate; its chemical formula is C₂₃H₄₄O₅, and its chemical structure is:

CH₃(CH₂)₇CH(OH)CH(OCOCH₂CH₂CH₃)(CH₂)₇COOCH₃.

Not illustrated separately is a second preferential form of Type B when the single branch is alternatively a nine-carbon chain length fatty acid [15] at carbon 10; that resulting molecule's scientific name is methyl 10-hydroxyoctadecanoate 9-nonanoate; its chemical formula is C₂₈H₅₄O₅, and its chemical structure is:

CH₃(CH₂)₇CH(OH)CH(OCO(CH₂)₇CH₃)(CH₂)₇COOCH₃.

FIG. 1C illustrates the third of three structural forms of the class of molecules that are to be synthesized, Type C. Type C has as its central skeleton [19] a form of stearic acid (scientific name, octadecanoic acid), an 18:0 carbon chain. This molecule differs from pure stearic acid as it also incorporates as part of the core carbon chain not just attached single hydrogen molecules but also a first branch containing a five-to-nine carbon chain length fatty acid [17] at carbon 9 and a second branch containing a five-to-nine carbon chain length fatty acid [21] at carbon 10. The resulting molecule's product name when it preferentially has a five carbon chain length fatty acid for each of the first and second branches is methyl octadecanoate 9,10-dibutyrate; its chemical formula is C₂₇H₅₀O₆; and its chemical structure is:

CH₃(CH₂)₇CH(OCOCH₂CH₂CH₃)CH(OCOCH₂CH₂CH₃)(CH₂)₇COOCH₃.

FIGS. 2A and 2B illustrate the two step synthesis from a methyl linoleate of a Type A form, or of branched methyl linoleate with butyric acid according to the method of the present invention. FIG. 2A shows the first step, in which an intermediate molecule [25] is produced from methyl linoleate [22] through epoxidation [23] using H₂O₂ and formic acid [42] to split each of the double carbon bonds, using each pair of freed carbon bonds to attach an additional atom of O. This reaction can be carried out in the standard way by the slow addition of HCO₃H (prepared from 35% H₂O₂ (20 mL) and HCO₂H (125 mL) at 0° C.) followed by stirring for 8 hours at 40° C. and then stirring at room temperature overnight. The mixture is distilled in vacuo (10 mm) and the residue is diluted with water and extracted with ether. FIG. 2B shows the second step, when through esterification of the intermediate molecule [25], using butyric acid, R₃N, and CH₃OH [44], the 5-to-9 carbons chain length molecules are attached, each attached O of the intermediate compound becomes an OH group, and the 5 or 9 carbons chain length molecules branching is attached adjacent to them, producing the branched methyl linoleate with butyric acid [29]. The esterification may be achieved using tertiary amine in the presence of methanol, as organic compounds are well known to form an ester with monocarboxylic acid. Azelaic acid can be obtained by oxidative cleavage of the carbon-carbon double bond through ozonolysis, and one equivalent of epoxidized methyl linoleate and two equivalents of monocarboxylic acid are required to get the desired branched molecule.

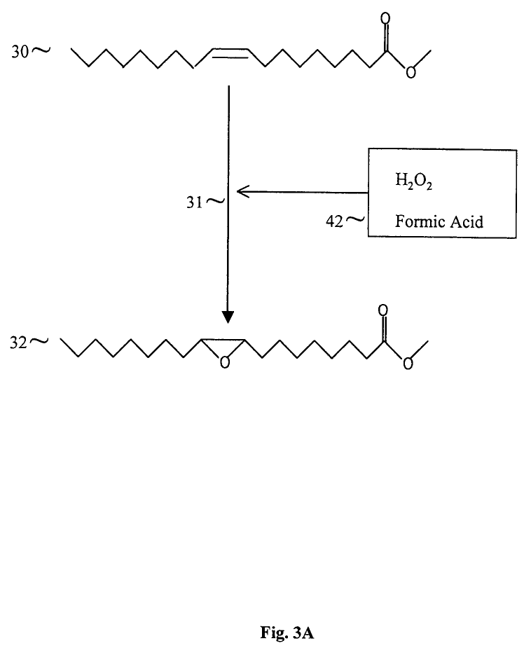

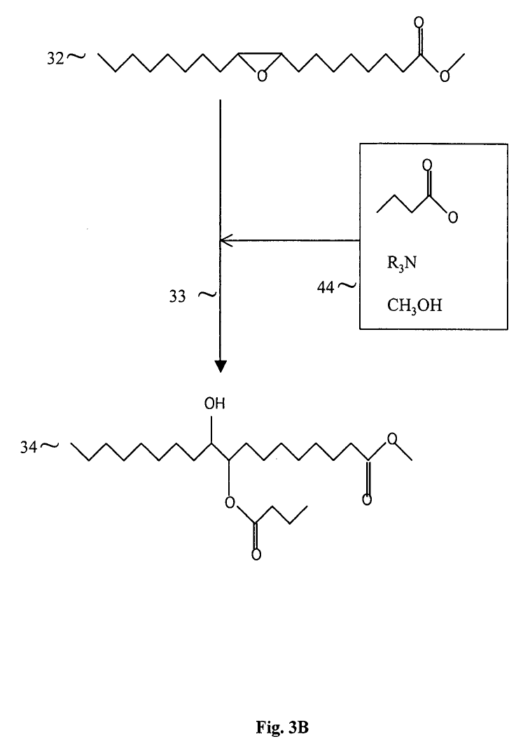

FIGS. 3A and 3B illustrate the two-step synthesis from methyl oleate of a Type B form, the first disclosed above, that is, a butyric of methyl oleate according to the method of the present invention. FIG. 3A shows the first step, in which an intermediate molecule [32] is produced from methyl oleate [30] through epoxidation [31] using H₂O₂ and formic acid [42] to split the double carbon bond, using the pair of freed carbon bonds to attach an additional atom of O. This reaction can be carried out as disclosed above. FIG. 3B shows the second step, when through esterification of the intermediate molecule [32], using preferentially butyric acid, R₃N, and CH₃OH [44], the attached O becomes an OH group and a five carbon chain length fatty acid branching is attached adjacent to produce the butyric of methyl oleate [34]. Two equivalents of epoxidized methyl oleate and one equivalent of dicarboxylic acid are required to get the desired branched molecule. The ‘R’ is a tertiary amine (e.g. Et₃N listed above in the ‘chemicals required’). This reaction can be carried out as disclosed above.

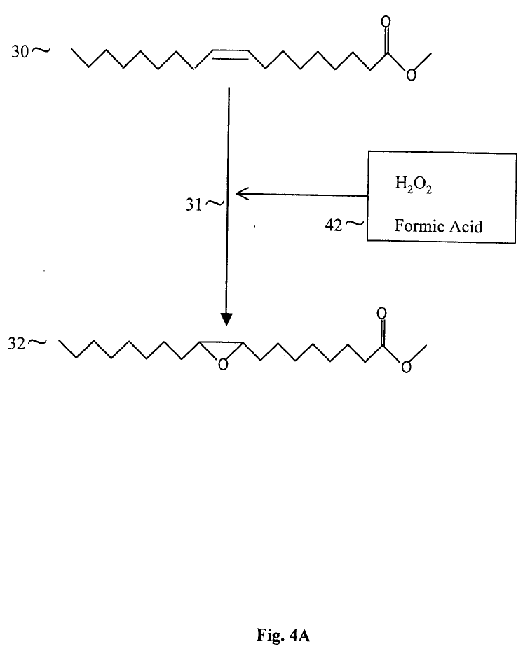

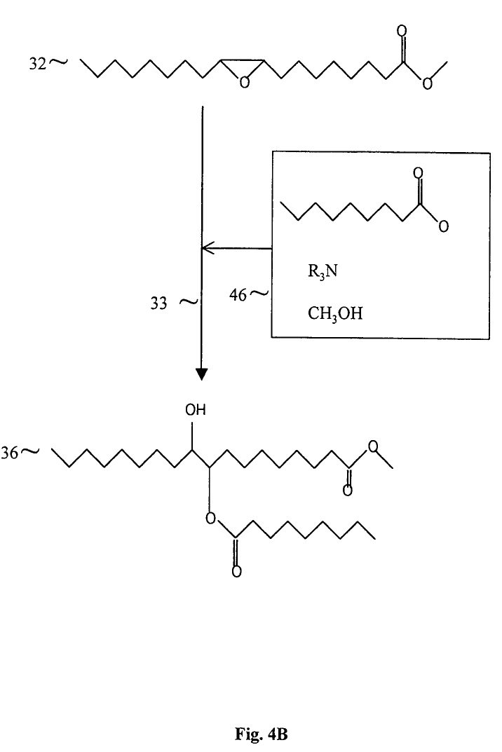

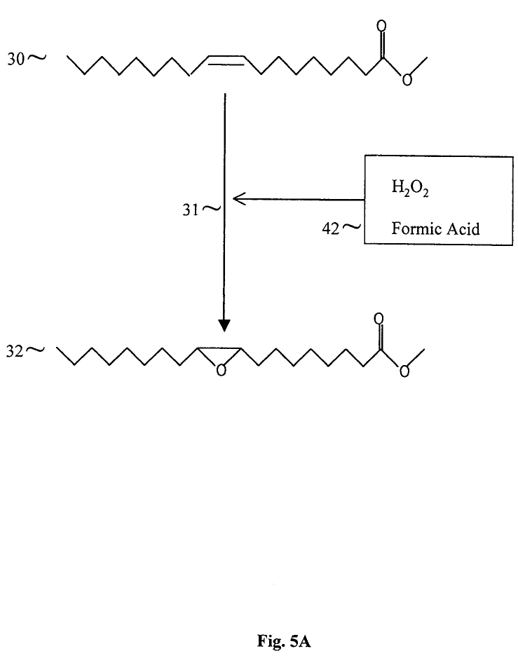

FIGS. 4A and 4B illustrate the two-step synthesis from methyl oleate of a Type B form, the second disclosed above, that is, a nonanoic of methyl oleate according to the method of the present invention. FIG. 4A shows the first step (the same as in FIG. 3A), in which an intermediate molecule [32] is produced from methyl oleate [33] through epoxidation [31] using H₂O₂ and formic acid [42] to split the double carbon bond, using the pair of freed carbon bonds to attach an additional atom of O. This reaction can be carried out as disclosed above. FIG. 4B shows the second step, when through esterification of the intermediate molecule [32], using preferentially nonanoic acid (a 9-carbon chain molecule), R₃N, and CH₃OH [46], the attached O becomes an OH group and a 9-carbons chain length molecules branching is attached adjacent, producing the nonanoic of methyl oleate [36]. This reaction can be carried out as disclosed above.

FIGS. 5A, 5B, and 5C illustrate the two-step synthesis from methyl oleate of a Type C form, that is, of a butryric anhydride according to the method of the present invention. FIG. 5A shows the first step (the same as in FIG. 3A and FIG. 4A), in which a first intermediate molecule [32] is produced from methyl oleate [30] through epoxidation [31] using H₂O₂ and formic acid [42] to split the double carbon bond, using the pair of freed carbon bonds to attach an additional atom of O. This reaction can be carried out as disclosed above. FIG. 5B shows the second step, where from the first intermediate molecule [32] through hydrolysis [37] using water (H₂O) and HClO₄ [44], a second intermediate molecule [38] is produced, in which two hydroxy groups are attached at the immediately adjacent carbons 9, 10. FIG. 5C shows the third step, where from the second intermediate molecule [38] through esterification [39] using butyric anhydride, BF₃, and Pyridine [48], an OH group is formed at carbons 9 and 12, and a five carbon chain fatty acid branching is attached adjacent and intervening at carbons 10 and 13 to produce the methyl oleate with butryric anhydride [40], where the OH groups make it polar and soluable in palmitic fatty acid. This reaction can be carried out as disclosed above.

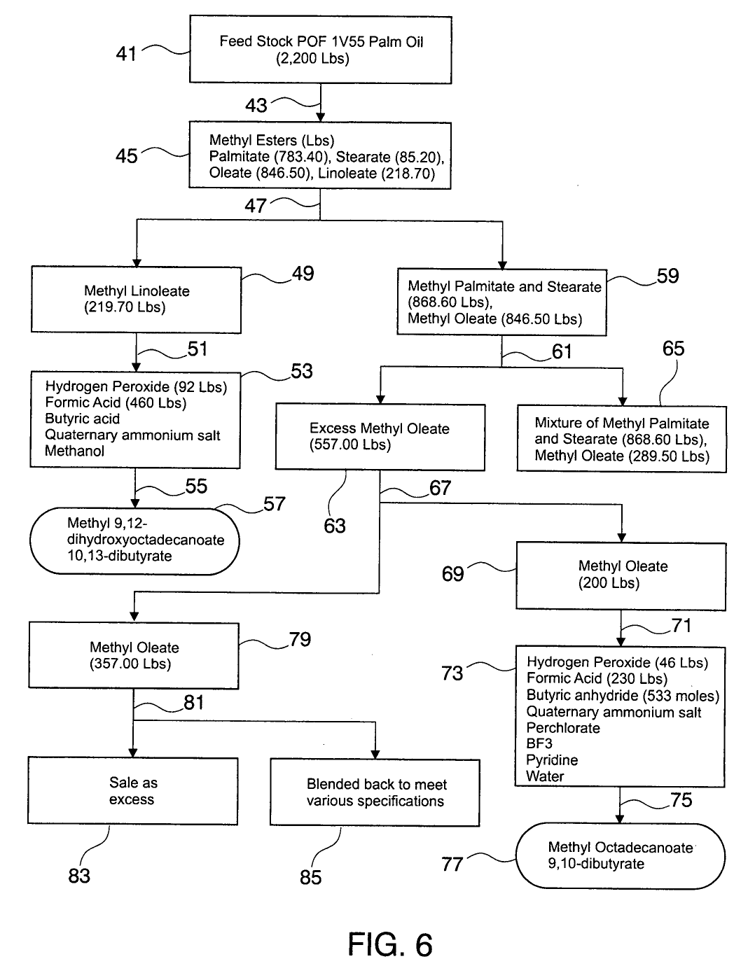

FIG. 6 is a flow chart showing how a single plant-oil feed stock containing varied fractions of plant oils (palmitic, oleic, stearic, linoleic, etc.) [41] which can be esterified [43] to yield a resulting percentage combination of varying forms of fatty acids (palmitate, oleate, stearate, linoleate, etc.] [45], which can be fractionated through standard separation processes [47]. The fractionated methyl linoleate [49] and the fractionated methyl palmitate, stearate, and methyl oleate [59] are separated. From the methyl linoleate [49], through the reactions disclosed above [51], using when necessary additional standard chemicals [53] that are removed [55], a Type A class of molecule that can serve as a subsequent base stock (shown here the preferred methyl 9,12-dihydroxyoctadecanoate 10,13-dibutyrate [57] can be synthesized. From the methyl palmitate, stearate, and methyl oleate [59], using standard separation processes [61], an excess of methyl oleate can be removed [63], leaving a combination of methyl palmitate and stearate and of methyl oleate in a 3:1 ratio [65]. This excess of methyl oleate can be further divided [67], with an unprocessed portion of it [79] further divided as desired [81] into amounts either being sold as excess [83] or blended back [85] with the other base stocks [57, 65, 77], or even returned to the excess [63] (this less-than-efficient ‘feedback loop’ is not shown). The other option for that methyl oleate which is further divided [67] is to be used, through the reactions disclosed above [71], using when necessary additional standard chemicals [73] that are removed [75], to form a Type B (not shown) or a Type C base stock, preferentially methyl octadecanoate 9,10-dibutyrate [77].

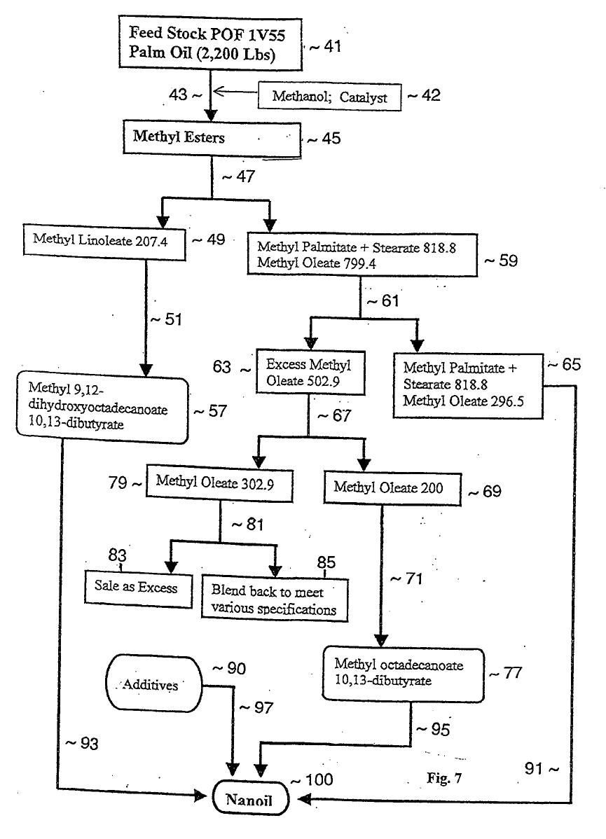

FIG. 7 is a modification of FIG. 6 showing the production of a plant-oil based lubricant [100] from the original plant-oil feed stock containing varied fractions of plant oils (palmitic, oleic, stearic, linoleic, etc.) [41]. The combination of methyl palmitate and stearate and of methyl oleate in a 3:1 ratio [65], a Type A feed stock, a Type C feed stock, and functional additives [90] are combined to form the plant-oil based lubricant [100] with properties determined according to the percentage blending of the compound; with the preferred embodiment using 60% by weight combined methyl palmitate and stearate and 20% by weight methyl oleate [91] (this alters the proportions of ‘excess’ and ‘combined’ methyl oleate, [63 and 65], 10% by weight the preferred Type A base stock methyl 9,12-dihidroxyoctadecanoate 10,13 butyrate [93], 9% by weight the preferred Type C base stock methyl octadecanoate 10,13 butyrate [95], and 1% by weight additives [97], thereby producing an entirely plant-oil based lubricant [100].

FIG. 7 thus is just one specific example (given the percentages and weights) disclosing an additional embodiment of the invention, where the final step is to combine the base stock (one of the class of molecules identified in FIG. 1 as Type A, Type B, and Type C) with esterified and fractionated saturated fats from a plant oil such as palm oil and additives, to create a blended composition that evinces the beneficial qualities of both saturated (high oxidative stability) and unsaturated (low, i.e. sub-zero F cloud or pour point), non-compounded and non-synthesized, pure plant oils. By varying the percentages of the base stocks, the specific plant oil(s) (whether saturated, unsaturated, or some admixture), and functional additives chosen, a wide range of desired characteristics can be obtained, enabling the production of products whose viscosity, viscosity index, pour point, oxidative stability, even flame point and biodegradation CEC rating, can be suited to the desired needs, without sacrificing the overall sourcing from renewable plant-oils.

Although the various aspects of the present invention have been described and exemplified above in terms of certain preferred embodiments, various other embodiments may be apparent to those skilled in the art. The invention is, therefore, not limited to the embodiments specifically described and exemplified herein, but is capable of variation and modification without departing from the scope of the appended claims.

I claim:

1. A method for synthesizing from unsaturated plant oils a class of molecules which can be compounded with saturated plant oils to obtain a resulting compound that possesses the beneficial properties of both saturated and unsaturated plant oils.

2. A method as in claim 1 further comprising compounding at least one of such class of molecules with saturated plant oils to obtain a resulting compound that possesses the beneficial properties of both saturated and unsaturated plant oils.

3. A method as in claim 1, comprising:
selecting a plant-oil based methyl linoleate;
attaining an intermediate molecule from the methyl linoleate through epoxidation, using H₂O₂ and formic acid to split each of the double carbon bonds in the methyl linoleate and attach an oxygen atom at each pair of carbons formerly sharing the double bond; and,
then synthesizing from the intermediate molecule through esterification a member of a class of molecules consisting of a variant from octadecanoic acid that attaches to the identified carbons, instead of single hydrogen molecules, at carbon 6 a first branch that is a five-to-nine carbon chain fatty acid, at carbon 7 a first hydroxy group, at carbon 9 a second branch that is also a five-to-nine carbon chain fatty acid, and at carbon 10 a second hydroxy group.

4. A method as in claim 3, wherein the step of attaining an intermediate molecule from the methyl linoleate through expoxidation further comprises:
preparing HCO₃H, by mixing 35% H₂O₂ (20 mL) and HCO₂H (125 mL) at 0° C.;
adding slowly HCO₃H to the methyl linoleate;
stirring the mixture of methyl linoleate and HCO₃H for 8 hours at 40° C.;
then stirring the mixture at room temperature overnight;
distilled the mixture in vacuo (10 mm);
diluting the residue with water; and,
extracting the intermediate molecule with ether.

5. A method as in claim 4, wherein the step of synthesizing from the intermediate molecule through esterification a member of a class of molecules consisting of a variant from octadecanoic acid that attaches to the identified carbons, instead of single hydrogen molecules, at carbon 6 a first branch that is a five-to-nine carbon chain fatty acid, at carbon 7 a first hydroxy group, at carbon 9 a second branch that is also a five-to-nine carbon chain fatty acid, and at carbon 10 a second hydroxy group, further comprises:
using a tertiary amine in the presence of methanol and the intermediate molecule to perform the esterification.

6. A method as in claim 3, wherein the step of synthesizing from the intermediate molecule through esterification a member of a class of molecules consisting of a variant from octadecanoic acid that attaches to the identified carbons, instead of single hydrogen molecules, at carbon 6 a first branch that is a five-carbon chain fatty acid, at carbon 7 a first hydroxy group, at carbon 9 a second branch that is also a five-carbon chain fatty acid, and at carbon 10 a second hydroxy group, thus creating methyl 9,12-dihydroxyoctadecanoate 10,13-dibutyrate.

7. A method as in claim 1, comprising:
selecting a plant-oil based methyl oleate;
attaining an intermediate molecule from the methyl oleate through epoxidation, using H₂O₂ and formic acid to split each of the double carbon bonds in the methyl linoleate and attach an oxygen atom at each pair of carbons formerly sharing the double bond; and,
then synthesizing from the intermediate molecule through esterification a member of a class of molecules consisting of a variant from octadecanoic acid that has at carbon 9 a hydroxy group and at carbon 10 a branch that is a five-to-nine carbon chain fatty acid.

8. A method as in claim 7, wherein the step of attaining an intermediate molecule from the methyl oleate through epoxidation, using H₂O₂ and formic acid to split each of the double carbon bonds in the methyl linoleate and attach an oxygen atom at each pair of carbons formerly sharing the double bond, further comprises:
preparing HCO₃H, by mixing 35% H₂O₂ (20 mL) and HCO₂H (125 mL) at 0° C.;
adding slowly HCO₃H to the methyl oleate;
stirring the mixture of methyl oleate and HCO₃H for 8 hours at 40° C.;
then stirring the mixture at room temperature overnight;
distilled the mixture in vacuo (10 mm);
diluting the residue with water; and,
extracting the intermediate molecule with ether.

9. A method as in claim 7, wherein the step of synthesizing from the intermediate molecule through esterification a member of a class of molecules consisting of a variant from octadecanoic acid that has at carbon 9 a hydroxy group and at carbon 10 a branch that is a five-to-nine carbon chain fatty acid, further comprises:
using a tertiary amine in the presence of methanol and the intermediate molecule to perform the esterification.

10. A method as in claim 7, wherein the step of synthesizing from the intermediate molecule through esterification a member of a class of molecules consisting of a variant from octadecanoic acid that has at carbon 9 a hydroxy group and at carbon 10 a branch that is a five-to-nine carbon chain fatty acid, further comprises:
using butyric acid, R₃N, and CH₃OH and the intermediate molecule to perform the esterification, to produce methyl 10-hydroxyoctadecanoate 9-butyrate.

11. A method as in claim 7, wherein the step of synthesizing from the intermediate molecule through esterification a member of a class of molecules consisting of a variant from octadecanoic acid that has at carbon 9 a hydroxy group and at carbon 10 a branch that is a five-carbon chain fatty acid, further comprises:
using nonanoic acid, R₃N, and CH₃OH and the intermediate molecule to perform the esterification, to produce methyl 10-hydroxyoctadecanoate 9-nonanoate.

12. A method as in claim 1, comprising:
selecting a plant-oil based methyl oleate;
attaining a first intermediate molecule from the methyl oleate through epoxidation, using H₂O₂ and formic acid to split each of the double carbon bonds in the methyl linoleate and attach an oxygen atom at each pair of carbons formerly sharing the double bond;
synthesizing from the first intermediate molecule, using hydrolysis using water and HClO₄, a second intermediate molecule in which two hydroxy groups are attached at the immediately adjacent carbons 9, 10; and,
then synthesizing from the second intermediate molecule through esterification a member of a class of molecules consisting of a variant from octadecanoic acid that has an OH group at each of carbons 9 and 12, and a five-to-nine carbon chain fatty acid branching attached at carbons 10 and 13.

13. A method as in claim 12, wherein the step of attaining a first intermediate molecule from the methyl oleate through epoxidation, using H₂O₂ and formic acid to split each of the double carbon bonds in the methyl linoleate and attach an oxygen atom at each pair of carbons formerly sharing the double bond, further comprises:
preparing HCO₃H, by mixing 35% H₂O₂ (20 mL) and HCO₂H (125 mL) at 0° C.;
adding slowly HCO₃H to the methyl oleate;
stirring the mixture of methyl oleate and HCO₃H for 8 hours at 40° C.;
then stirring the mixture at room temperature overnight;
distilled the mixture in vacuo (10 mm);
diluting the residue with water; and,
extracting the intermediate molecule with ether.

14. A method as in claim 12, wherein the step of synthesizing from the second intermediate molecule through esterification a member of a class of molecules consisting of a variant from octadecanoic acid that has an OH group at each of carbons 9 and 12, and a five-to-nine carbon chain fatty acid branching attached at carbons 10 and 13, further comprises:
using a tertiary amine in the presence of methanol and the intermediate molecule to perform the esterification.

15. A method as in claim 12, wherein the step of synthesizing from the second intermediate molecule through esterification a member of a class of molecules consisting of a variant from octadecanoic acid that has an OH group at each of carbons 9 and 12, and a five-to-nine carbon chain fatty acid branching attached at carbons 10 and 13, further comprises:
using butyric anhydride, BF₃, and Pyridine to produce a variant from octadecanoic acid that has an OH group at each of carbons 9 and 12, and a five-carbon chain fatty acid branching attached at each of carbons 10 and 13, thereby producing methyl octadecanoate 10,13 butyrate.

16. A base stock for a plant-oil based fuel, oil, or lubricant comprising any of the set of the following four molecules, the first of which is synthesized from the methyl form of linoleic acid and the remaing three of which are synthesized from the methyl form of oleic acid, according to the method disclosed in claim 1, said set consisting of:
methyl 9,12-dihydroxyoctadecanoate 10,13-dibutyrate;
methyl 10-hydroxyoctadecanoate 9-butyrate;
methyl 10-hydroxyoctadecanoate 9-nonanoate; and,
methyl octadecanoate 9,10-dibutyrate.

17. A method for synthesizing from unsaturated plant oils a class of molecules which can be compounded with saturated plant oils to obtain a resulting compound that possesses the beneficial properties of both saturated and unsaturated plant oils, comprising:
starting with a plant-oil base containing both saturated and unsaturated oils;
using esterification on the plant-oil base to produce saturated and unsaturated methyl esters;
synthesizing from a specific methyl ester a base stock with desired characteristics by inducing any of hydroxy groups and five-to-nine carbon chain branching on selected carbons of the specific methyl ester; and,
blending the base stock with the saturated and unsaturated methyl esters in varying proportions to produce a plant-oil based resulting product;
which resulting product may then be used as any of a fuel, oil, and lubricant.

18. A method as in claim 17, further comprising, between the steps of synthesizing from a specific methyl ester a base stock with desired characteristics by inducing any of hydroxy groups and five-to-nine carbon chain branching on selected carbons of the specific methyl ester and blending the base stock with the saturated and unsaturated methyl esters in varying proportions to produce a plant-oil based resulting product:
blending a proportion of the non-synthesized, saturated and unsaturated methyl esters wherein the proportion of methyl palmitate, methyl stearate, and methyl oleate each may range from being solely a third to solely a fifteenth of the total by weight.

19. A method as in claim 17, further comprising:
blending the base stock, the saturated unsaturated methyl esters, and unsaturated methyl esters in varying proportions with an additive, wherein the additive may range from zero to fifty percent by weight of the total blend.

20. A method as in claim 17, further comprising:
using a palm oil as the plant oil base;
producing from the palm oil methyl esters of palmitate, stearate, oleate, and linoleate;
using the linoleate to produce a first class of base stock;
blending the palmitate, stearate, and a portion of the methyl oleate to produce a second class of base stock, leaving a remainder of methyl oleate; and,
using a portion of the remainder of methyl oleate to produce a third class of base stock.

21. A method as in claim 20, wherein the step of blending the palmitate, stearate, and a portion of the methyl oleate to produce a second class of base stock, leaving a remainder of methyl oleate further comprises:
blending equal portions of methyl palmitate and stearate with the portion of methyl oleate in a ratio between 1.6:1 and 20:1, by weight.

22. A method as in claim 17, further comprising:
combining a portion of the first class of base stock, a portion of the second class of base stock, and a portion of the third class of base stock, to produce a plant-oil based fuel, oil or lubricant with the desired functional characteristics.

23. A method as in claim 22, further comprising adding an additive, wherein the additive may range from zero to 50% by weight of the total blend.

24. A method as in claim 23, wherein:
the first class of base stock comprises between 2 and 15%, by weight, of the final product;
the second class of base stock comprises between 40 and 80%, by weight, of the final product;
the third class of base stock comprises between 2 and 15%, by weight, of the final product; and,
an additive comprises between zero and 50% by weight, of the final product; and, where the total of first class of base stock, second class of base stock, third class of base stock, and additive, equals 100% of the weight of the final product.
